(19) United States
(12) Patent Application Publication
(10) Pub. No.: US 2022/0233816 A1
(43) Pub. Date: Jul. 28, 2022

(54) CATHETER HUB ADAPTED TO BE USED WITH MULTIUSE BLOOD CONTROL VALVE

(71) Applicant: Smiths Medical ASD, Inc., Plymouth, MN (US)

(72) Inventors: James M Muskatello, Southington, CT (US); David J Goral, Brookfield, CT (US)

(73) Assignee: Smiths Medical ASD, Inc., Plymouth, MN (US)

(21) Appl. No.: 17/612,593

(22) PCT Filed: Jun. 23, 2020
(86) PCT No.: PCT/US2020/039134

§ 371 Date: Nov. 19, 2021

Related U.S. Application Data
(60) Provisional application No. 62/866,951, filed on Jun. 26, 2019.

(51) Int. Cl. A61M 25/00; A61M 25/06; A61M 39/24

(52) U.S. Cl. CPC A61M 25/0097; A61M 39/24; A61M 25/0074; A61M 25/0606

(57) ABSTRACT

A catheter hub of a safety needle assembly that includes a multiuse valve uses a proximal portion of the catheter as an actuator to open the partition membrane of the valve when the valve is subjected to an external biasing force. In another embodiment, the actuator is formed as an integral component of the catheter hub.

This is a US National stage application of PCT Application No. PCT/US2020/039134, filed Jun. 23, 2020, which in turn claims priority from U.S. Provisional application No. 62/866,951 filed Jun. 26, 2019. The contents of the '134 and '951 applications are fully incorporated herein by reference.

### FIELD OF THE INVENTION

The instant invention relates to intravenous catheter assemblies and other vascular access devices including peripheral intravenous catheter (PIVC) assemblies and, more particularly, to an improved catheter hub adapted to be used with a resealable valve to control the blood flow of the catheter assemblies.

### BACKGROUND OF THE INVENTION

Catheter assemblies typically have a catheter hub that has a catheter tube extending from a distal end of the catheter hub. A needle cannula or simply needle slidably extends through the lumen of the catheter so that the sharp tip of the needle extends beyond the distal end of the catheter. The catheter is guided into the appropriate vasculature for example a vein or artery of a patient by the sharp tip of the needle inserting into the patient and then the vasculature. Once the catheter is in place, the needle is removed from the catheter and the catheter hub. Thereafter, a fluid store device such as a syringe or pump would be coupled to the catheter hub so that a fluid path is established between the vasculature of the patient for example a vein and the fluid store device by way of the catheter and the interior space of the catheter hub.

To ensure that the catheter is correctly positioned into the vein, or artery, of the patient, the needle may have a side opening that allows blood to seep into the space between the internal surface of the catheter and outer circumferential surface of the needle. This blood along the catheter may act as an indication to the clinician that the catheter is correctly placed. However, after the removal of the needle from the catheter but before the coupling of the fluid store device to the catheter hub, pressured blood may undesirably back flow into the catheter hub via the unobstructed lumen of the catheter.

To prevent the blood back flow, the prior art discloses an elastomeric septum or seal member provided in the catheter hub. The stationary seal member is responsive to the insertion movement of a probe for example the male luer taper into the cavity of the catheter hub and opens when the luer lock or end connector of the fluid store device is firmly coupled to the luer end of the catheter hub. The fluid store device may be a syringe, an administration set or a pump for example.

Patents and applications assigned to the assignee of the instant invention describe a compressible valve that is biased by the inserted luer taper against an actuator fixedly attached to the interior of the catheter hub. The actuator is a separate component that has an eyelet portion. To attach the actuator and the catheter to the catheter hub, the eyelet portion is press fitted into the aperture at the distal end of the catheter along with the proximal end of the catheter. In order to be able to press fit into the aperture, at least the eyelet portion of the actuator is made of a metallic material. Thus, in addition to requiring separate components, catheter assemblies having the actuator press fitted catheter hub may not be safely used in magnetic resonance imaging (MRI). The patents and applications assigned to the assignee of the instant application that describe a catheter hub that has the compressible valve and the fixedly attached actuator include: U.S. Pat. Nos. 8,652,104, 9,399,116, 9,545,495, 10,080,867 and U.S. application Ser. Nos. 16/110,051 and 16/110,111. The respective disclosures of the '104, '116, '495 and '867 patents and the '051 and '111 applications are incorporated by reference herein to the disclosure of the instant application.

### SUMMARY OF THE PRESENT INVENTION

The instant invention is directed to an improved catheter hub for use with a catheter assembly. Instead of a separate actuator, in a first embodiment of the instant invention, the catheter is used to form both the catheter and the actuator of the catheter hub. To that end, a catheter is fittingly threaded through the aperture at the distal end of the catheter hub so that a distal portion extends distally away from the catheter hub and a proximal portion extends proximally into the internal cavity of the catheter hub. The portion of the catheter sandwiched by the distal and proximal portions is fixedly bonded to the aperture by any of the conventional methods including for example chemical adhesive bonding, melt bonding, plastic insert molding, among others. The proximal portion of the catheter may be configured to be structurally rigid and has the appropriate columnar strength so that its probe end is adapted to open the resealable partition or membrane of a multiuse valve. This may be achieved by injection molding the catheter so that the thickness of the circumferential wall of the proximal portion is greater than the thickness of the circumferential wall of the distal portion. alternatively, the proximal and distal portions of the catheter may be extruded with different material, or have particles added to the proximal portion to add columnar strength and rigidity thereto. The proximal portion of the catheter that extends into the cavity of the catheter hub may be referred to as the catheter actuator or simply the actuator.

An elastomeric resealable multiuse valve that has a partition or membrane that separates the valve into a proximal portion and a distal portion is slidably and non-removably inserted into the cavity of the catheter hub with its membrane proximal to the actuator when the valve is in its natural unbiased state. The membrane has at least one slit to assist in its opening. Multiple slits may be scored to the membrane to provide flaps when the membrane is opened. At the unbiased position, the flexibility of the membrane is such that the membrane remains closed to prevent fluid leak from the portion of the catheter hub closed off by the valve even when the catheter is positioned in the artery of the patient and the blood in the lumen of the catheter is under pressure.

The membrane is biased against the actuator to an opened position in the catheter hub when the contact end of the connector (luer taper of the luer lock connector) of an external device for example a fluid store device comes into contact with the proximal end of the valve, as the external fluid store device is coupled to the catheter hub. With the membrane opened, an open fluid communication path is established between the chamber at the proximal portion of the valve, the cavity at the proximal portion of the catheter hub, and the lumen of the catheter, so that fluid may be collected from, or infused to, the patient. When the external fluid store device is removed from the catheter hub, no longer under bias and due to its inherent elasticity, the valve returns to its natural state with the membrane again positioned proximal to the probe end of the actuator in the closed position.

Another embodiment of the present invention has an actuator integrally formed as a part of the catheter hub. For this embodiment, the actuator integrally extends from an internal distal end wall inside the catheter hub as a tubular structure having an internal passage that aligns with the aperture at the distal end of the catheter hub. The actuator may have a frusto-conical probe end to assist in the opening of the membrane. The proximal portion of the catheter may extend into and fixedly attach to the aperture at the distal end of the catheter hub. Alternatively, the proximal portion of the catheter may extend through the passage of the actuator so that the lumen of the catheter provides a through passageway between the internal cavity of the catheter hub and the vein or artery of the patient.

With the various embodiments of the present invention, there is no need for a separate actuator component and the additional process of installing the separate actuator component into the catheter hub. Cost savings thus result both from the lesser number of components as well as the lesser amount of manufacturing required to produce the catheter hub assembly. Further, by eliminating the metallic actuator, the inventive catheter assembly may be safely used in MRI scans.

### BRIEF DESCRIPTION OF THE FIGURES

The present invention will become apparent and the invention itself will be best understood with reference to the following description of the invention taken in conjunction with the accompanying drawings, wherein:

FIG. 1 is a semi-transparent perspective rear view of a first embodiment of the catheter hub assembly of the instant invention;

FIG. 2 is a semi-transparent perspective front view of the first embodiment of the catheter hub assembly of the instant invention;

FIG. 3 is a disassembled view of the catheter hub assembly in the first embodiment of the instant invention;

FIG. 4 is a cut away cross-sectional view of the catheter hub of the first embodiment of the instant invention;

FIG. 5 is a semi-cut away cut away cross-sectional view of the catheter hub assembly of the first embodiment of the instant invention;

FIG. 6 is a semi-cut away of the outer wall perspective view of the catheter hub assembly of the first embodiment;

FIG. 7 is a perspective semi-transparent view of the catheter hub assembly and the multiuse valve positioned therein;

FIG. 8 is a cross-sectional view of the instant inventive catheter hub assembly and its relationship to the multiuse valve in an unbiased position;

FIG. 9 is a cross-sectional view of the first embodiment of the instant inventive catheter hub assembly and the positioning of the multiuse valve relative to the actuator portion of the catheter when the valve is in a biased position;

FIGS. 10A and 10B are cross-sectional views of the inventive catheter hub assembly showing the membrane of the multiuse valve in its natural closed position and its opened position, respectively;

FIG. 11 is a cross-sectional view of another embodiment where the actuator portion of the catheter inside the cavity of the catheter hub is flared or enlarged and is located distal to the closed membrane of the multiuse valve;

FIG. 12 is a cross-sectional view of the catheter hub of FIG. 11 showing the multiuse valve being biased to its opened position by the flared catheter actuator;

FIGS. 13A and 13B illustrate the positioning of the multiuse valve relative to the flared catheter actuator with the membrane in a closed position and an opened position, respectively;

FIGS. 14A, 14B and 14C are respective cross-sectional views of different exemplar multiuse valves adapted to be used in the catheter hub assembly of the instant invention;

FIG. 15 is a cut away view of a further embodiment of the catheter hub of the instant invention;

FIG. 16 is a cross-sectional view showing the catheter hub of FIG. 15 with a catheter; and

FIG. 17 is a cross-sectional view showing an embodiment where the proximal portion of the catheter is extended through the internal passage of the integral actuator of the catheter hub.

### DETAILED DESCRIPTION OF THE INVENTION

For the description below, the terms proximal and non-patient, and the terms distal and patient, when used to denote the direction of the inventive catheter assembly, are interchangeable. For example, the terms distal end and patient end denote the same direction, while the terms proximal end and non-patient end denote the same opposite direction.

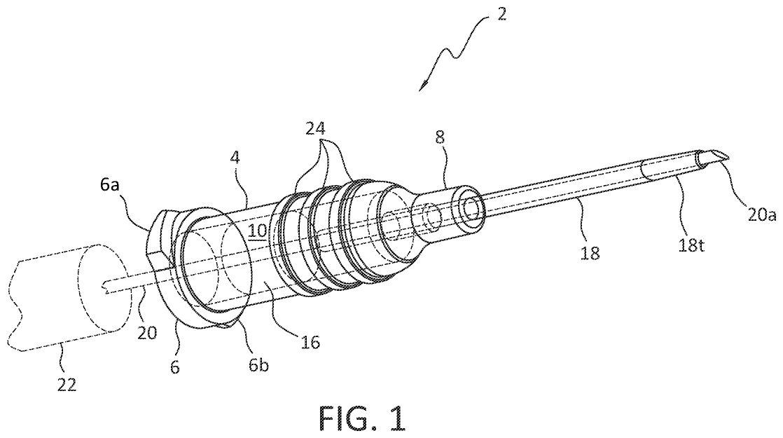

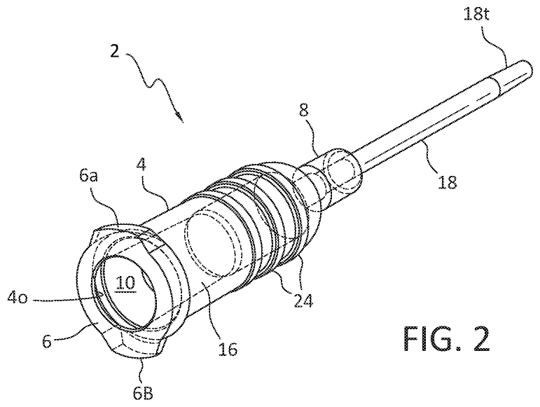

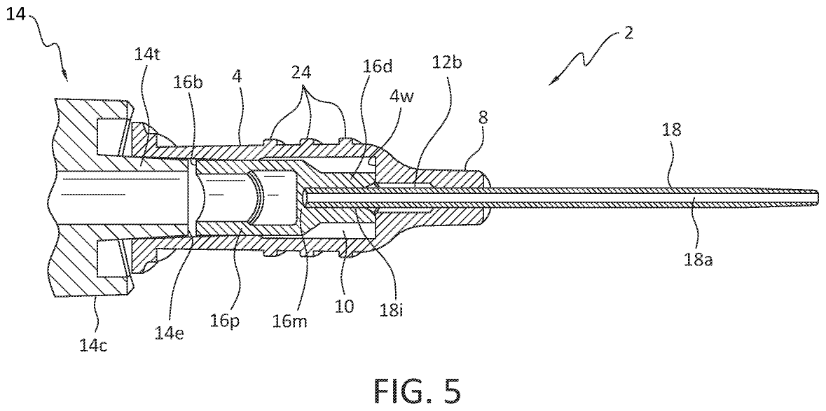

With reference to FIGS. 1-5, a first embodiment of a catheter hub assembly 2 of the instant invention is shown to have an elongated cylindrical body 4 that has an opened proximal end 6, a distal end 8 and an internal cavity 10 that opens to opening 4o. Cavity 10 has a proximal section 4p and a slightly larger diameter distal section 4d separated by a transition 4s. An aperture 12 extends through distal end 8. Although not required for the embodiment, aperture 12 is shown to have a distal portion 12a and a proximal portion 12b, with portion 12b having a cross section greater than that of portion 12a. Proximal end 6 has two tabs 6c and 6b that enable the proximal end 6 to lockingly couple with a luer connector 14c of an external device 14, shown in FIG. 5 and represented by directional arrow 14 in other figures. The external device may be a fluid store device including for example a syringe, an administration set or a pump or the like. When thus coupled, the luer taper cone or simply luer taper 14t of the external device enters into the cavity 10 of the catheter hub into contact with the proximal end 16b of a multiuse valve 16 positioned in the catheter hub, as will be described in greater detail below. Luer taper 14t may also be referred to as the connector end of the external device.

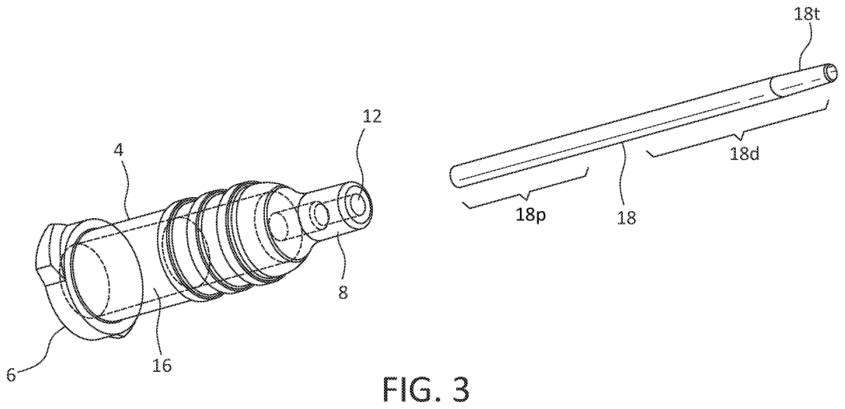

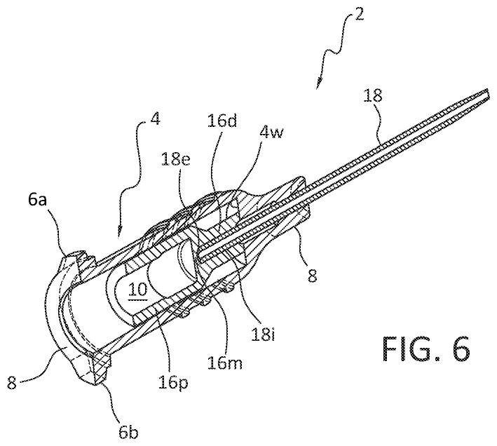

A catheter 18 extends from the distal end 8 of the catheter hub. As best shown in FIG. 3, catheter 18 has a proximal portion 18p and a distal portion 18d having a distal tip 18t. As shown in FIG. 5, the proximal portion 18p of catheter 18 is extended through aperture 12 into the internal cavity 10 of catheter hub 4. Once catheter 18 is slidably fitted through aperture 12, a portion of proximal portion 18p is fixedly attached to the inner wall at distal end 8 of catheter hub 4 that defines aperture 12 by any of a number of conventional attachment methods including for example gluing, adhesive bonding, melt bonding, ultrasonic bonding or the like. Aperture 12 may be configured into two sections 12a and 12b with catheter 18 slidably form fitted through section 12a and adhesive provided to section 12b to fixedly bond catheter 18 to the catheter hub. The length of the portion of catheter 18 that extends proximally into cavity 10 is designated 18i in FIG. 6, and may be referred to as the actuator portion of the catheter, the catheter actuator or simply actuator. Actuator 18i may be configured to have a frusto-conical probe end 18e to assist in the opening of a membrane in valve 16. The length of actuator 18i inside cavity 10 is dependent on the rigidity and columnar strength of at least that portion of the catheter required to act as the actuator to open the membrane or partition of valve 16, as well as the length and cross dimension of the portion of the valve 16 that prevents fluid leak from the catheter actuator, as will be further described infra.

Catheter 18 may be extruded to have different materials or mixture of materials so that the rigidity and columnar strength for the actuator portion is stronger than the rest of the catheter. Catheter 18 may also be formed by injection molding with the material that forms the actuator portion having more rigidity than the material that forms the patient end of the catheter. Materials that may be used for extruding or molding catheter 18 may include polyurethane or nylon, and other conventionally known materials. Air bubbles and other non-metallic particles may also be added to the actuator portion during the extrusion or molding process to provide different columnar strengths for the different portions of the catheter. Also, the wall of the actuator portion may be formed to have a greater thickness than the wall for the patient portion of the catheter.

Valve 16, which may also be referred to as a seal member, is an elongated cylindrical member made of an elastomeric material such as for example silicone or polyisoprene, or other similar materials that have the required flexibility and compressibility characteristics. One exemplar multiuse valve 16, as shown in FIGS. 5-7, has a proximal portion 16p and a distal portion 16d. Valve 16 has a cross dimension that enables it to slidably fit into the cavity 10 of the catheter hub. A through passage 16o extends between a proximal end 16b and a distal end 16e of valve 16 to form a chamber inside valve 16. A notch 16n at the outer surface of proximal portion 16p provides an outlet for air to escape from inside cavity 10 when the distal portion 16d is compressed in the catheter hub. Notch 16n also provides an inlet for sterilization gas to pass into distal portion of the cavity of the catheter hub to sterilize the catheter hub assembly. The inherent elasticity characteristics of the elastomer material enables the valve to compress inside cavity 10 when it encounters a biasing force along its longitudinal axis, and returns to its natural state when the biasing force is removed. It should be appreciated that distal portion 16d may function more as the compressible biasing member of valve 16 since it can more readily compress in response to a biasing force due to the space in cavity 10 that surrounds it, and then decompresses to return the valve to its natural state once the biasing force is removed.

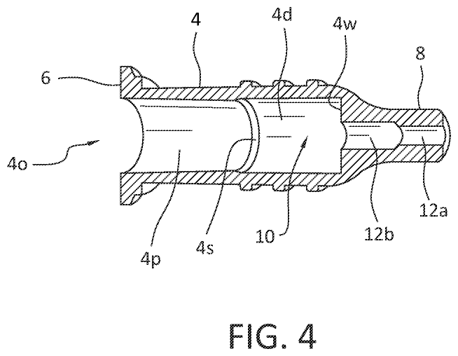

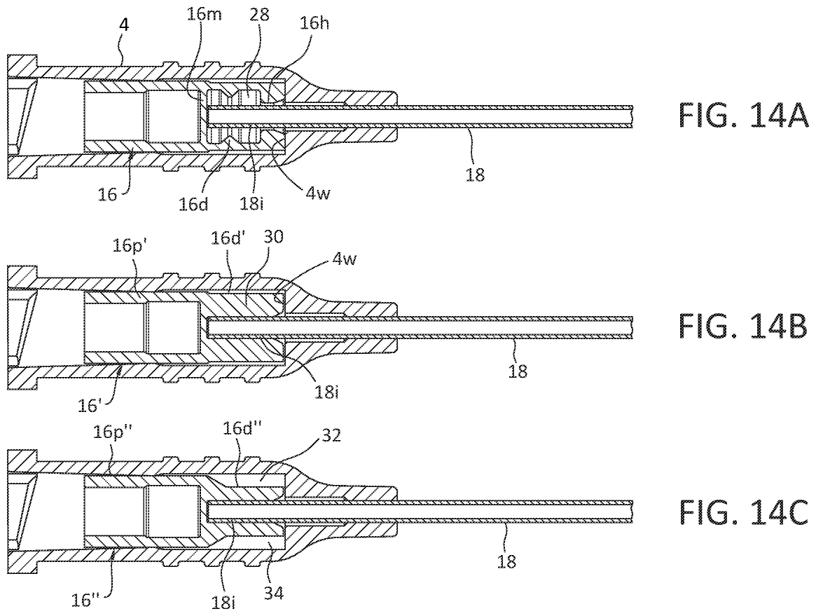

An integral membrane or partition 16m of valve 16 separates proximal portion 16p and distal portion 16d. Distal end 16e of valve 16 is in contact with an inside distal wall 4w of catheter hub 4. Distal wall 4w defines the distal end of the internal cavity 10 of catheter hub 4. As best shown in FIG. 4, wall 4w encircles aperture 12. Although wall 4w is shown to be flat in the exemplar embodiment, it should be appreciated that wall 4w has a counterpart configuration adapted to reflect the particular or given configuration of distal end 16e of valve 16, so as to ensure that the maximum contact area is provided between wall 4w and distal end 16e to enable optimal compression by valve 6 under a biasing force and the return of valve 6 to its natural position when the biasing force is removed. For example, if the distal end 16e has a rounded configuration, then wall 4w would have a counterpart grooved configuration to receive distal end 16e. Or if the distal end 16e is flat, then distal wall 4w likewise is flat. Even though the distal portion for the exemplar valve embodiment may be considered as the biasing member, It should be appreciated that, as noted above, due to the inherent elasticity of the elastomer material, the entire valve may act as one biasing member, as will be described later with respect to the embodiment shown in FIG. 14B.

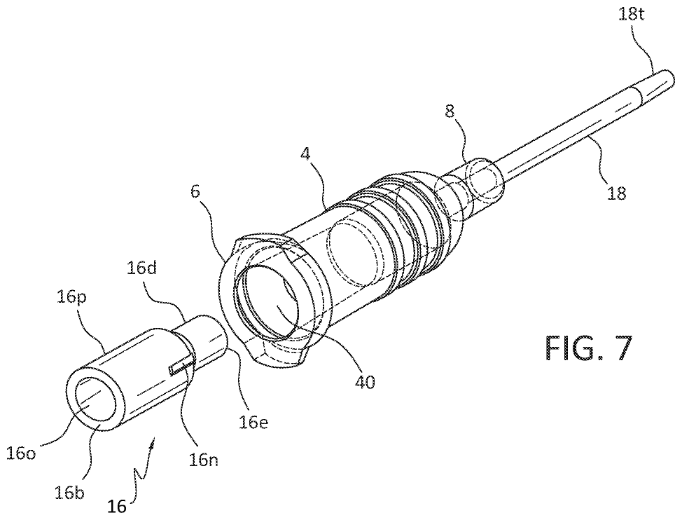

As shown in FIG. 7, valve 16 is slidably inserted into cavity 10 of catheter hub through opening 4o at proximal end 6 of catheter hub 4. Cooperating means for example cooperating groove(s) and flange(s), not shown, may be provided at the outer surface of valve 16 and the inner circumferential surface of catheter hub 4, respectively, to maintain valve 16 inside but slidable within catheter hub 4.

With reference to FIG. 1, in a ready to use position, a needle cannula, or simply needle 20, extending from a needle hub or a needle insertion device or assembly 22, is inserted into cavity 10 of catheter hub 4, through membrane 16m of valve 16, and slidably extends through lumen 18a of catheter 18 with its sharp tip 20a extending beyond the distal end 18t of catheter 18.

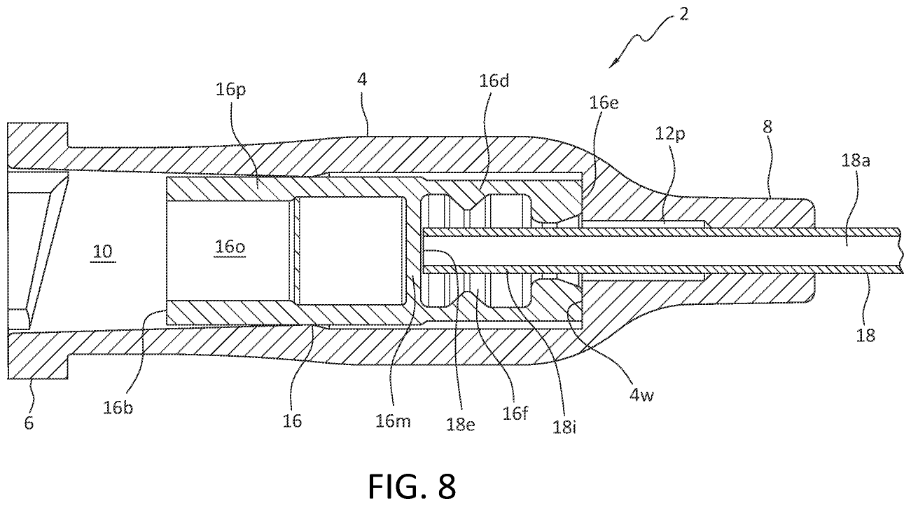

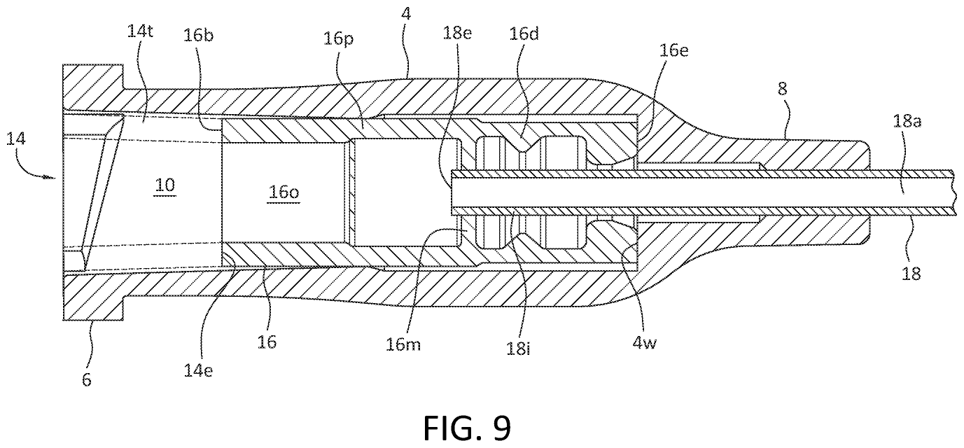

FIGS. 8 and 9 are illustrations of another exemplar multiuse valve adapted to be used with the catheter assembly of the instant invention. Components that are the same as the exemplar valve described above are labeled with the same reference numbers. As shown, the distal section 16d of the multiuse valve 16 of the FIGS. 8-9 embodiment has an outer circumferential surface that is a substantial continuation of the outer circumferential surface of proximal section 16p, while its inside surface is unevenly formed to define a chamber 16f that houses catheter actuator 18i. Distal portion 16d is the biasing portion of the exemplar valve 16 as it can compress inwardly into chamber 16f. Valve 16 shown in FIGS. 8-9 is similar to the seal member described in the afore-noted incorporated by reference patents and publications, for example U.S. Pat. No. 8,652,104. FIG. 8 shows valve 16 in its natural state where membrane 16m is proximal of probe end 16e of actuator 18i.

FIG. 9 shows an external device, designated by arrow 14, being coupled or connected to proximal luer end 6 of catheter hub 4. As the external device connects to proximal end 6 with its luer lock, its luer taper 14t is inserted into cavity 10 of the catheter hub and its distal connector end 14e comes into contact with proximal end 16b of valve 16. Due to its elasticity, when biased by luer taper 14t, valve 16, particularly its distal portion 16d, compresses against distal end wall 4w inside catheter hub 4, as described in greater detail in the above-noted incorporated by reference patents and publications, such as for example the disclosure with reference to FIGS. 9, 10 and 19 in the '104 patent. Further distal movement of the distal connector end 14e moves valve 16 in the distal direction relative to catheter hub 4 to bias membrane 16m against actuator 18i, more particularly its probe end 18e until the opening of probe end 18e is positioned in through passage 16o, which may also be referred to as chamber or proximal chamber 16o, of valve 16. As a result, an open fluid communication passageway is established between the external fluid store device and the vein of the patient through lumen 18a of catheter 18, chamber 16o of valve 16 and cavity 10 inside catheter hub 4. It should be appreciated that since chamber 16o and cavity 10 are both inside catheter hub 4, the term cavity may also define the internal space or cavity of the catheter hub open to proximal probe end 18e of actuator 18i. When the external device is removed from catheter hub 4 thereby removing the force biasing membrane 16m against actuator 18i, valve 6 returns to its natural state or unbiased position where membrane 16m is closed and is proximal of probe end 18e, as per shown in FIG. 8. To assist in the opening and closing of membrane 16m, as discussed above, one or more slits may be formed in membrane 16m.

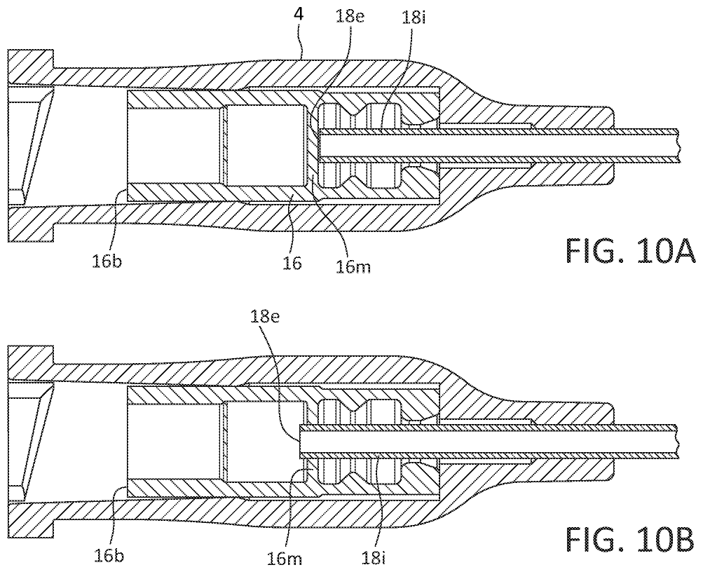

FIGS. 10A and 10B illustrate the distal movement of valve 16 and shows membrane 16m in the closed state and the opened state, respectively.

With reference to FIG. 1, to use the catheter assembly of the instant invention, the clinician pierces into the vasculature for example a vein or a blood vessel of the patient by using the sharp tip 20a of needle 20. Needle 20 is then moved to position catheter 18 that overlays needle 20 in the vein. After catheter 18 is correctly positioned, the needle is removed so that the catheter assembly is as per shown in the cross section view of FIG. 8. The clinician may use the number of suture tie down rings 24 at the outer surface of catheter hub 4 and tape to attach the catheter hub assembly to the skin of the patient. A fluid store device for example a syringe may then be connected to the catheter assembly by coupling the luer connector of the fluid store device to the luer end 6 of catheter hub 4.

After the removal of the needle 20 from the catheter hub assembly 2 but before the coupling of the external device to catheter hub 4, catheter assembly 2 is as per illustrated in FIG. 8 where valve 16 is in its natural state. As shown, membrane 16m is closed and positioned proximal to probe end 18e of catheter actuator 18i. At this position, membrane 16m partitions the proximal chamber 16o at proximal portion 16p from the distal chamber 16f at distal portion 16d of valve 16. With membrane closed, blood back flow from the patient through lumen 18a of catheter 18 is prevented from escaping chamber 16f.

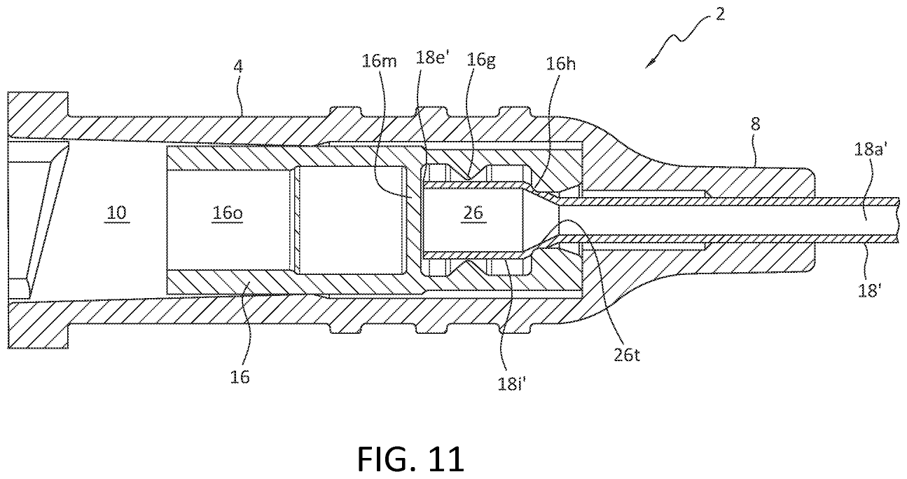

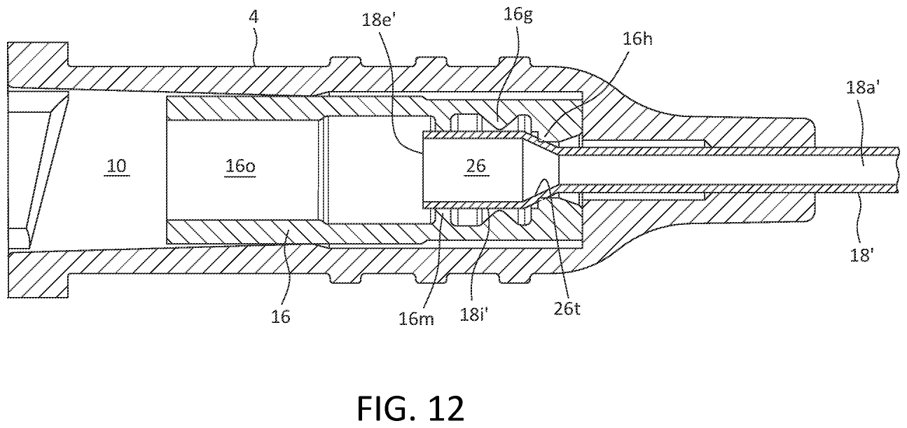

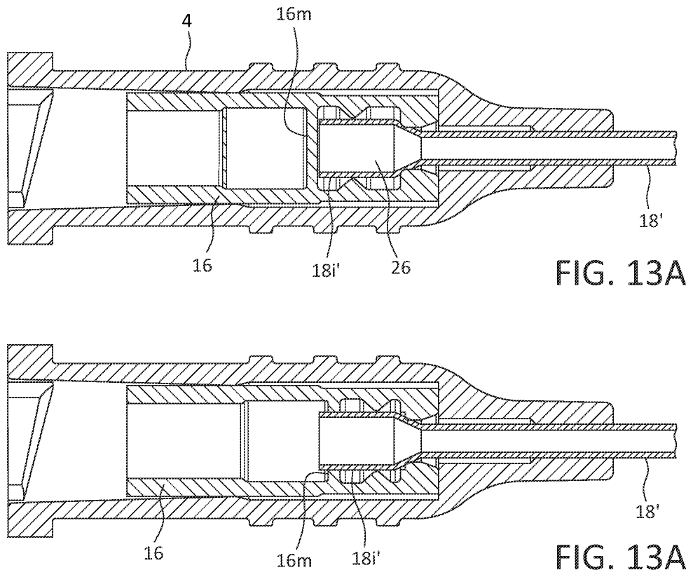

FIGS. 11 and 12 illustrate another embodiment that uses a one piece catheter as both the actuator and the patient vasculature conduit is shown. The same reference numbers in FIGS. 1-10 used to identify the same components in the being discussed figures are repeated. As shown, the actuator 18i′ of the catheter 18′ is enlarged by a flaring device so that the flared cylindrical catheter actuator 18i′ has a bore 26 that tapers to meet lumen 18a′ via a tapered section 26t. By having a flared actuator, membrane 16m, when biased against probe end 18e′, has a larger opening. This may lead to more fluid flow through the catheter assembly. Also, the tapered section 26t is holdingly supported by the circumferential protrusion 16h while the flared portion 18i′ is holdingly supported by circular protrusion 16g of valve 16 to provide a more stable actuator. The operation and the function of the catheter assembly shown in FIGS. 11 and 12 are the same as discussed above for the embodiment shown in FIGS. 1-10. FIGS. 13A-13B show the movement of the valve relative to the catheter hub when the membrane is not biased against the flared actuator and is in its unbiased closed position and when the membrane is biased against the flared actuator and is in its opened position.

With reference to FIGS. 14A-14C, three embodiments of the multiuse valve adapted to be used with the actuator in the catheter hub are shown. The different multiuse valves are shown to be in their unbiased position. Components that are the same as those described in the earlier figures are labeled with the same reference numbers. The multiuse valve 16 shown in FIG. 14A is the same valve that is described in the incorporated by reference '104 patent. Cavity 28 in the distal portion 16d of valve 16 enables distal portion 16d to be readily compressed against distal end wall 4w when valve 16 is biased in the distal direction relative to the catheter hub 4. The circumferential protrusion 16h provides support for catheter actuator 18i.

FIG. 14B shows a multiuse valve 16′ that has a distal portion 16d′ formed with a substantially solid elastomeric portion 30 in support of actuator 18i. For the embodiment, the elastomeric material that forms portion 30 may be a material that has more elasticity or flexibility such as for example Elastosil that allows portion 30 to be compressed more readily. For the FIG. 14B embodiment, even though shown to have the same length as the catheter actuators in FIGS. 14A and 14B, the length of the catheter actuator 18i inside the catheter hub may be varied (shortened or lengthened) as discussed above and the length of distal portion 30 of the valve may also be varied to balance its elasticity against the distance to end wall 4w that is needed for the membrane of the valve to be opened by actuator 18i under a biasing force. Further, the proximal portion 16p′ of the valve may be strengthened with additives, or enforced or replaced for example by a cylindrical ring having a higher shore hardness than the elastomeric material that the valve is made from.

FIG. 14C shows a multiuse valve 16″ that was shown in FIGS. 5-7. Valve 16″ has a distal portion 16d″ that tapers from the distal end of the proximal portion 16p″ so that distal portion 16d″ has a smaller diameter than the diameter of proximal portion 16p″. Accordingly, there is a circumferential space 32 surrounding the elongated cylinder 34 of the distal portion 16d″ of the valve. In this embodiment, cylindrical cylinder 34 provides support for the entire length of the catheter actuator 18i and space 32 provides the room for cylinder 34 to be folded or compressed when the valve is moved distally by a biasing force.

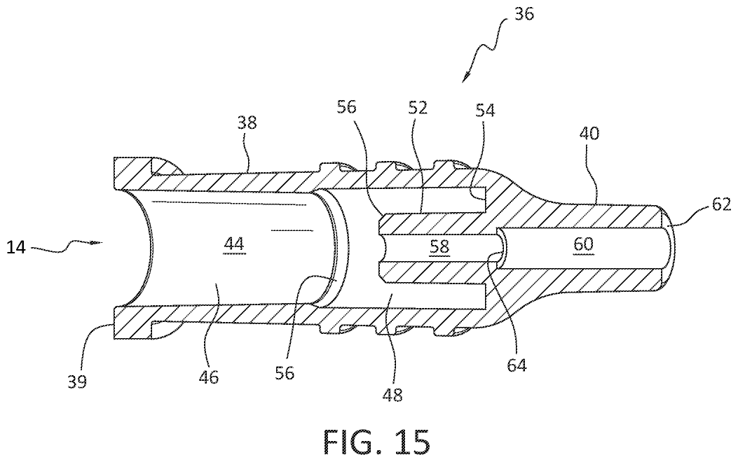

Another embodiment of the inventive catheter hub is illustrated in FIGS. 15-17. As shown, catheter hub 36 has an elongated cylindrical body and a distal end 40 that tapers from body 38 and a proximal end 39 that is configured to accept a luer connector from an external device as per discussion above. Similar to the earlier discussed embodiments, catheter hub 36 has a proximal end 42 that opens to an internal cavity 44 that has a smaller diameter proximal section 46 and a larger diameter distal section 48 connected by a tapered transition 50. Instead of utilizing the proximal portion of the catheter to be the actuator, an actuator 52 of this embodiment extends integrally from the inside distal end wall 54 of the catheter hub. In other words, actuator 52 is an integral component of the one piece catheter hub 36 that may be formed from the same mold. As shown, actuator 52 is a cylindrical extension that has a frusto-conical probe end 56 and a passage 58 in alignment with aperture 60 at distal end 40 of catheter hub 36. Passage 58 and aperture 60 together form a through passageway from distal end 62 to internal cavity 10. Although shown not to have the multiuse valve, it should be appreciated that the multiuse valve 16 described in the earlier embodiments may be provided in cavity 44 of catheter hub 36 and be utilized in the same manner, i.e., a distal force, designated by arrow 14, moves the valve in the distal direction relative to the catheter hub so that the membrane 16m of the valve is biased against probe end 56 of actuator 52 to its opened position; and when the external distal force is removed, the valve would return to its natural state so that its membrane is proximal to the actuator 52 in its closed position to prevent blood leak. By having an integral actuator, there is no need for the type of support that may be needed for a catheter actuator as described in the earlier embodiment. Manufacturing cost may also be reduced due to the reduction in the manufacturing steps as the actuator and the catheter hub are molded as a one piece component.

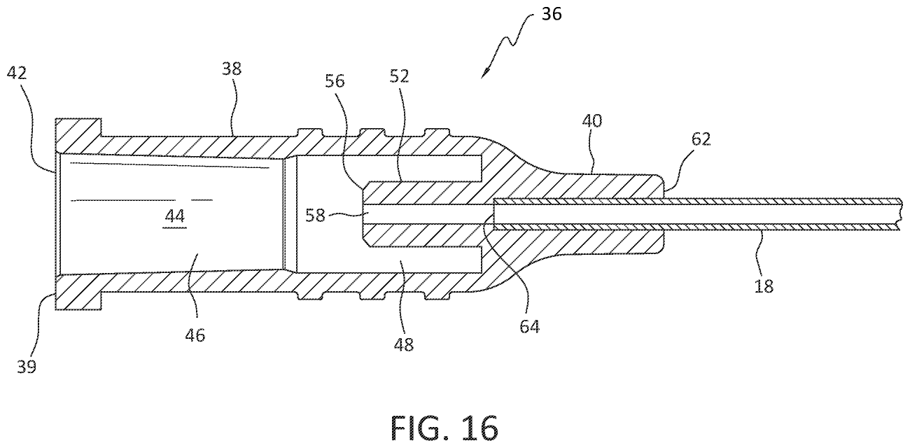

FIG. 16 shows a catheter 18 slidably fitted into aperture 60. A step 64 that joins aperture 60 and passage 58 acts as a stop to the proximal end of the catheter. The portion of catheter 18 in aperture 60 is fixedly attached to the inside circumferential surface of the aperture by any of the conventional bonding methods described previously. With the catheter attached to the catheter hub 36, a fluid communication path is established from the distal end of the catheter to cavity 44 and to the external fluid store coupled to the proximal end of the catheter hub.

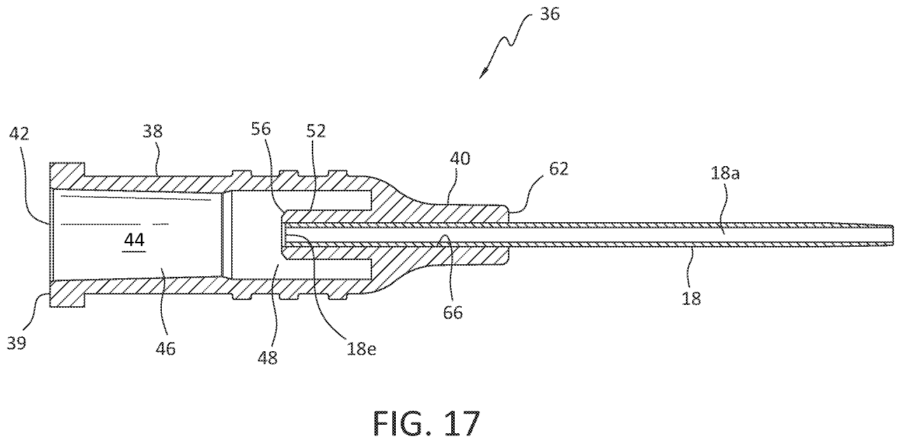

FIG. 17 is an illustration of an embodiment in which a through aperture 66 extends from distal end 62 of the catheter hub 36 to probe end 56 of actuator 52. A proximal portion of catheter 18 is slidably form fitted into aperture 60′ to probe end 56 of actuator 52. The catheter may be bondedly attached to aperture 66 as described above, or pressure fitted into aperture 66. By extending the catheter through to probe end 56 of actuator 52, a continuous lumen of the same diameter is provided.

The invention disclosed above is subject to many variations, modifications and changes in detail. Thus, it is intended that all matters described throughout this specification and shown in the accompanying drawings be interpreted as illustrative only and not in a limiting sense. Accordingly, it is intended that the invention be limited only by the spirit and scope of the hereto attached claims.

1. A catheter hub, comprising:
a body having a distal end and a proximal end, the body having a cavity opened at the proximal end and an aperture at the distal end, the cavity extending from the opened proximal end to an inside distal wall encircling the aperture at the distal end, a catheter having a proximal portion, a distal portion and a through lumen fittingly extending through the aperture such that the distal portion of the catheter is distally external of the catheter hub and an actuator defined by the proximal portion of the catheter extends proximally inside the cavity of the catheter hub.

2. The catheter hub of claim 1, wherein the actuator of the catheter inside the cavity has a probe end for opening a resealable partition of a valve positioned in the cavity when the valve is biased distally relative to the catheter hub such that the partition comes into contact with the probe end and is opened thereby, the lumen establishing a through passageway between the proximal end of the catheter hub and a distal end of the catheter.

3. The catheter hub of claim 2, wherein the proximal portion of the catheter that defines the actuator is configured to have a rigidity and columnar strength sufficient to open the partition.

4. The catheter hub of claim 1, wherein the cavity is substantially in the shape of a cylinder and the inside distal wall that forms the distal end of the cavity encircling the aperture has a counterpart configuration to a given configuration of the distal end of the valve that contacts the inside distal wall.

5. The catheter hub of claim 1, wherein the proximal portion of the catheter inside the cavity has a flared cylindrical portion that includes a probe end.

6. The catheter hub of claim 1, further comprising:
a valve positioned in the cavity, the valve having a resealable partition, a proximal valve end and a distal valve end, the distal valve end in contact with the inside distal wall and the proximal valve end adapted to be biased by a contact end of an external device, the partition located proximal of a probe end of the actuator when the valve is in an unbiased state.

7. The catheter hub of claim 6, wherein when the contact end of the external device makes contact with the proximal valve end and moves the valve distally relative to the catheter hub, the partition is opened by the probe end of the actuator so that an open passageway is established through the lumen of the catheter between the proximal end of the catheter hub and a distal end of the catheter.

8. The catheter hub of claim 6, further comprising:
a needle passing through the partition and slidably extending through the lumen of the catheter such that a distal tip of the needle extends beyond a distal end of the catheter when the catheter hub is in a ready to use position.

9. A catheter hub, comprising:
a body having a distal end, an opened proximal end and an internal cavity between the proximal end and a distal wall inside the cavity encircling an aperture at the distal end, a hub portion of a catheter having a lumen fittingly accepted in the aperture, an actuator integrally formed with the body extending away from the distal wall into the cavity, the actuator having an internal passage in alignment with the aperture so as to be in fluid communication with the lumen of the catheter.

10. The catheter hub of claim 9, further comprising:
a valve positioned in the cavity, the valve having a proximal valve end, a distal valve end and a resealable partition, the distal valve end in contact with the distal wall, the partition being proximal of a probe end of the actuator when the valve is in an unbiased state.

11. The catheter hub of claim 10, wherein the cavity is substantially in the shape of a cylinder except for the actuator extending into the cavity from the distal wall, the distal end of the valve having a given configuration, and the distal wall surrounding the actuator has a counterpart configuration to the given configuration for receiving the distal end of the valve.

12. The catheter hub of claim 10, wherein when an external device is coupled to the catheter hub, the valve is moved by the external device in a distal direction to bias the partition against the probe end of the actuator to establish an open passageway between the proximal end of the catheter hub and the lumen of the catheter through the internal passage of the actuator.

13. The catheter hub of claim 11, further comprising:
a needle slidably extending through the partition, the internal passage of the actuator and the lumen of the catheter such that a distal tip of the needle extends beyond a distal end of the catheter when the catheter hub is in a ready to use position.

14-20. (canceled)

21. A method of making a catheter hub, comprising the steps of:
providing a body having a distal end, a proximal end, a cavity opened at the proximal end and an aperture at the distal end, the cavity extending from the opened proximal end to an inside distal wall encircling the aperture at the distal end;
providing a catheter having a proximal portion, a distal portion and a through lumen;
fittingly extending the catheter through the aperture such that the distal portion of the catheter is distally external of the catheter hub and an actuator defined by the proximal portion of the catheter extends proximally inside the cavity of the catheter hub.

22. The method of claim 21, further comprising the step of:
inserting a valve having a resealable partition in the cavity;
wherein the actuator of the catheter inside the cavity has a probe end for opening the resealable partition of the valve when the valve is biased distally relative to the body such that the partition comes into contact with the probe end and is opened thereby to establish a through passageway between the proximal end of the body and a distal end of the catheter.

23. The method of claim 21, further comprising the step of:
configuring the proximal portion of the catheter inside the cavity to have a flared cylindrical portion that includes a probe end.

24. The method of claim 22, further comprising the step of:
configuring the cavity of the body to be substantially in the shape of a cylinder; and
configuring the inside distal wall that forms the distal end of the cavity encircling the aperture to have a counterpart configuration to a given configuration of the distal end of the valve that contacts the inside distal wall.
